(19) United States
(12) Patent Application Publication
(10) Pub. No.: US 2008/0114054 A1
(43) Pub. Date: May 15, 2008

(54) Compositions and methods for reducing antimicrobial resistance of microbes

(76) Inventors: Rath Microbes, Heerlen (NL); Aleksandra Niedzwiecki, San Jose, CA (US); Steve Harakeh, Baabda (LB)

Correspondence Address: INHOUSE CO., 280 COLORADO AVE., PALO ALTO, CA, 94301, US

(21) Appl. No.: 11/599,661

(22) Filed: Nov. 14, 2006

(51) Int. Cl. A61K 31/401

(52) U.S. Cl. 514/428

(57) ABSTRACT

The instant methods and compositions represent an improvement in reducing antimicrobial resistance of microbes and enhancing antimicrobial activity of drugs. A composition comprising of polyphenols, an ascorbic compound, L-lysine, L-proline, and L-arginine have been found to reduce the antimicrobial resistance of microbe. As such, methods of treating infection, and methods of enhancing antimicrobial activity of drugs are provided herein.

### FIELD OF THE INVENTION

This invention relates to compositions and methods for treating health conditions caused by an infection and methods for reducing antimicrobial resistance of bacteria for the purpose of enhancing antimicrobial activities of drugs.

### BACKGROUND OF THE INVENTION

Antibiotic resistance is now recognized as a pressing public health problem. Antibiotics, also known as antimicrobial drugs, are drugs that fight infections caused by bacteria. Unfortunately, antibiotic use promotes development of antibiotic-resistance in bacteria. Antibiotic resistance occurs when bacteria change in some way to reduce or even eliminate the effectiveness of drugs, compositions and agents intended to treat health conditions caused by an infection.

Since their initial discovery in 1929, antibiotics have extensively transformed the health care industry's approach to infectious disease. The use of antibiotics, combined with improvements in sanitation, nutrition, and implementation of effective vaccination programs, have led to a systematic reduction in common infectious diseases. Terrifying infectious diseases such as plague, whooping cough, polio and scarlet fever, which once struck terror in the minds of millions, are now under control. Nonetheless, once again we are faced with the crisis of infectious diseases as microbes develop resistance to antimicrobials. Curable diseases such as gonorrhea and typhoid are now becoming or have become difficult to treat. Furthermore, once notorious infectious diseases, such as tuberculosis and malaria, are now flourishing again under the shield of antimicrobial resistance.

Bacteria often become resistant to antimicrobial agents. This is possible by bacteria's acquisition of genes that encode enzymes to inactivate an agent or by the bacteria's modification of the target agent. Indeed, enzymes that inactivate synthetic antibiotics such as quinolones, sulfonamides, and trimethoprim have not been discovered. Instead, resistance usually arises against these synthetic antibiotics by target agent modifications. Accordingly, antibiotic resistance can cause significant damage and suffering to children and adults having infections that were once easily treatable.

As such, methods for enhancing drugs antimicrobial activity and reducing antimicrobial resistance in bacteria as well as improved methods for treating health conditions caused by infection are desperately needed.

U.S. Pat. No. 5,872,104 describes using a methylation inhibitor that inhibits methylation and maturation of bacterial RNA, thereby reducing the resistance of bacteria and other microorganisms to antimicrobial agents. U.S. Pat. Nos. 6,346,391 and 6,677,133 describe a method of treating an infections caused by a drug resistant microbe using compounds that block the function of AcrAB-like pump thereby reducing the drug resistance of bacteria. PCT Pat. Appln. Pub. No. WO 03/028762 A1 describes a composition that controls or reduces the antimicrobial resistance of bacteria using a compound that blocks development of bacterial resistance. All of the above patents are hereby incorporated in full by reference.

### SUMMARY OF THE INVENTION

The present invention is based, at least in part, on the discovery that a composition comprising lysine, proline, arginine, an ascorbic compound, and polyphenols will reduce antimicrobial resistance of microbes thereby enhancing the efficacy of antimicrobials used.

Accordingly, in one aspect, the invention provides methods for preventing or treating health conditions caused by an infection by administering to a mammal one or more drugs in conjunction with a composition comprising lysine, proline, arginine, an ascorbic compound, and polyphenols in the effective amount.

In one embodiment, the composition is comprised of lysine, proline, arginine, ascorbic acid, calcium, magnesium, polyphenols, N-acetyl-cysteine, selenium, copper, EGCg, and manganese. In another embodiment, the composition is comprised of approximately 400-1500 mg lysine, 500-1500 mg proline, 200-1000 mg arginine, 400-1500 mg of ascorbic compound, 5-50 mg calcium, 10-100 mg magnesium, 500-2000 mg polyphenols, 100-500 mg N-acetyl-cysteine, 5-60 μg selenium, 0.5-5 mg copper, and 0.5-3 mg manganese. In a preferred embodiment, the composition is comprised of approximately 1000 mg lysine, 750 mg proline, 500 mg arginine, 710 mg of ascorbic compound, 22 mg calcium, 50 mg magnesium, 800 mg polyphenols, 200 mg N-acetyl-cysteine, 30 μg selenium, 2 mg copper, and 1 mg manganese.

In one embodiment, the drugs are antibiotics. In a preferred embodiment the antibiotics are selected from the groups consisting of a Ciprofloxacin (CIP), a Trimethoprim-Sulfamethoaxzole (SXT), a Cefotaxime (CTX), a Gentamicin (GM), an Ofloxacin (OFX), a Nalidixic acid (NA), an Ampicillin (AMP), a Vancomycin (V), a Tetracycline (TE), a Rifampicin (RA), a Doxycycline (D), a Ceftriaxone (Cro), and a Kanamycin (K).

In another embodiment, the composition is provided in the form of a tablet, a pill, an injection, an infusion, an inhalation, or a suppository. In yet another embodiment, the composition is provided in a pharmaceutically accepted carrier and means of delivery.

In another aspect, the invention provides method of treating an infection comprising the step of administering to a mammal an effective amount of a composition comprising lysine, proline, arginine, ascorbic compound, and polyphenols. In a preferred embodiment, the composition is comprised of lysine, proline, arginine, ascorbic compound, calcium, magnesium, polyphenols, N-acetyl-cysteine, selenium, copper, and manganese.

In one embodiment, the composition is comprised of lysine, proline, arginine, ascorbic acid, calcium, magnesium, polyphenols, N-acetyl-cysteine, selenium, copper, EGCg, and manganese. In another embodiment, the composition is comprised of approximately 400-1500 mg lysine, 500-1500 mg proline, 200-1000 mg arginine, 400-1500 mg of ascorbic compound, 5-50 mg calcium, 10-100 mg magnesium, 500-2000 mg polyphenols, 100-500 mg N-acetyl-cysteine, 5-60 μg selenium, 0.5-5 mg copper, and 0.5-3 mg manganese. In a preferred embodiment, the composition is comprised of approximately 1000 mg lysine, 750 mg proline, 500 mg arginine, 710 mg of ascorbic compound, 22 mg calcium, 50 mg magnesium, 800 mg polyphenols, 200 mg N-acetyl-cysteine, 30 μg selenium, 2 mg copper, and 1 mg manganese.

In yet another aspect, the invention provides a method of enhancing the antimicrobial activity of a drug comprising treating a microbe that is resistant to one or more drugs with a drug to which the microbe is resistant to in conjunction with a composition comprising lysine, proline, arginine, an ascorbic compound, and polyphenols in the effective amount. In a preferred embodiment, the composition is comprised of lysine, proline, arginine, ascorbic compound, calcium, magnesium, polyphenols, N-acetyl-cysteine, selenium, copper, and manganese.

In one embodiment, the composition is comprised of lysine, proline, arginine, ascorbic acid, calcium, magnesium, polyphenols, N-acetyl-cysteine, selenium, copper, EGCg, and manganese. In another embodiment, the composition is comprised of approximately 400-1500 mg lysine, 500-1500 mg proline, 200-1000 mg arginine, 400-1500 mg of ascorbic compound, 5-50 mg calcium, 10-100 mg magnesium, 500-2000 mg polyphenols, 100-500 mg N-acetyl-cysteine, 5-60 μg selenium, 0.5-5 mg copper, and 0.5-3 mg manganese. In a preferred embodiment, the composition is comprised of approximately 1000 mg lysine, 750 mg proline, 500 mg arginine, 710 mg of ascorbic compound, 22 mg calcium, 50 mg magnesium, 800 mg polyphenols, 200 mg N-acetyl-cysteine, 30 μg selenium, 2 mg copper, and 1 mg manganese.

In one embodiment, the drugs are antibiotics. In a preferred embodiment the antibiotics are selected from the groups consisting of a Ciprofloxacin (CIP), a Trimethoprim-Sulfamethoaxzole (SXT), a Cefotaxime (CTX), a Gentamicin (GM), an Ofloxacin (OFX), a Nalidixic acid (NA), an Ampicillin (AMP), a Vancomycin (V), a Tetracycline (TE), a Rifampicin (RA), a Doxycycline (D), a Ceftriaxone (Cro), and a Kanamycin (K).

In another embodiment, the composition is provided in the form of a tablet, a pill, an injection, an infusion, an inhalation, or a suppository. In yet another embodiment, the composition is provided in a pharmaceutically accepted carrier and means of delivery.

Further yet in another aspect, the invention provides a pharmaceutical composition comprising an antibiotic and a composition comprising lysine, proline, arginine, ascorbic acid, calcium, magnesium, polyphenols, N-acetyl-cysteine, selenium, copper, and manganese. In a preferred embodiment, the antibiotic is selected from the group consisting of a Ciprofloxacin (CIP), a Trimethoprim-Sulfamethoaxzole (SXT), a Cefotaxime (CTX), a Gentamicin (GM), an Ofloxacin (OFX), a Nalidixic acid (NA), an Ampicillin (AMP), a Vancomycin (V), a Tetracycline (TE), a Rifampicin (RA), a Doxycycline (D), a Ceftriaxone (Cro), and a Kanamycin (K).

In another embodiment, the composition is provided in the form of a tablet, a pill, an injection, an infusion, an inhalation, or a suppository. In yet another embodiment, the composition is provided in a pharmaceutically accepted carrier and means of delivery.

### BRIEF DESCRIPTION OF THE DRAWING

FIG. 1 shows an increase in the inhibition zone of Salmonella bacterial colonies after exposing the colonies to a composition in accordance with the invention comprising list of components and amounts during the exponential growth stage in the presence of antibiotics.

FIG. 2 shows an increase in susceptibility of Salmonella bacterial colonies to the antimicrobial effect of an antimicrobial agent after exposing the colonies to a biochemical composition in accordance with the invention comprising list components and amounts during the exponential growth stage in the presence of antibiotics.

FIG. 3 shows an increase in the inhibition zone of S. aprophtocus bacterial colonies after exposing the colonies to a composition in accordance with the invention comprising list of components and amounts during the exponential growth stage in the presence of antibiotics.

FIG. 4 shows an increase in the inhibition zone of S. aureus bacterial colonies after exposing the colonies to a composition in accordance with the invention comprising list of components and amounts during the exponential growth stage in the presence of antibiotic.

FIG. 5 shows an increase in the inhibition zone of Listeria bacterial colonies after exposing the colonies to a composition in accordance with the invention comprising list of components and amounts during the exponential growth stage in the presence of antibiotics.

FIG. 6 show an increase in the inhibition zone of Brucella bacterial colonies after exposing the colonies to a composition in accordance with the invention comprising list of components and amounts during the exponential growth stage in the presence of antibiotics.

FIG. 7 show an increase in the inhibition zone of Yerssinia bacterial colonies after exposing the colonies to a composition in accordance with the invention comprising list of components and amounts during the exponential growth stage in the presence of antibiotics.

FIG. 8 shows an increase in the inhibition zone of E. Coli bacterial colonies after exposing the colonies to a composition in accordance with the invention comprising list of components and amounts during the exponential growth stage in the presence of antibiotics.

### DETAILED DESCRIPTION OF THE INVENTION

The term “infection” as used herein refers to the presence of a microbe in or on a subject which, if growth of the microbe were inhibited, would result in a benefit to the subject. Accordingly, the term “infection” refers to both the presence of pathogens and undesirable normal flora at any anatomical site.

The term “treating” as used herein refers to the administration of a compound to a subject for therapeutic purposes. The term “administration” includes delivery to a subject by any appropriate method which serves to deliver the drug to the site of the infection. The administration of drug can be oral, nasal, parentral, topical, ophthalmic, or transdermal administration or delivery in the form of solid, semi-solid, lyophilized powder, or liquid dosage forms. The dosage forms include tablets, suppositories, pills soft elastic and hard gelatin capsules, powders, solutions, suspensions, or aerosols, or the like, preferably in unit dosage forms suitable for simple administration of precise dosages.

The term “drug” as used herein includes antibiotic agents and non-antibiotic agents. Such drugs include compounds which reduce the proliferation of a microbe, or compounds which reduce the ability of a microbe to effectuate infection in a host, or compounds which reduce the ability of a microbe to multiply or remain infective in an environment. The term “drug” also includes antiinfective compounds which are static or cidal for microbes, or an antimicrobial compound which will inhibit the growth of a microbe. Preferred antiinfective compounds increase the susceptibility of microbes to antibiotics or decrease the infectivity of a microbe.

The term “drug” as used herein also includes an antimicrobial agent that has been shown to mediate resistance in a multiple antibiotic resistance phenotype. The term “drug” therefore, includes disinfectants, antiseptics, and surface delivered compounds. The term “drug” further includes biocidal agents. The term “biocidal” is recognized and includes an agent that one of ordinarily skill in the art would recognize as capable of killing a cell “non-specifically,” or a broad spectrum agent whose mechanism of action is unknown.

The term “antibiotic” as used herein includes antimicrobial agents which are synthesized by an organism in nature and chemically synthesized thereby. The term includes but is not limited to: Ciprofloxacin (CIP), a Trimethoprim-Sulfamethoaxzole (SXT), a Cefotaxime (CTX), a Gentamicin (GM), an Ofloxacin (OFX), a Nalidixic acid (NA), an Ampicillin (AMP), a Vancomycin (V), a Tetracycline (TE), a Rifampicin (RA), a Doxycycline (D), a Ceftriaxone (Cro), and a Kanamycin (K). Likewise, semi-synthetic derivatives of antibiotics, and antibiotics produced by chemical methods are also encompassed by this term. Chemically-derived antimicrobial agents such as Trimethoprim are considered antibacterial drugs, and the term antibiotic includes these.

The term “microbes” as used herein refers to a microorganism, preferably unicellular microbes including bacteria including any prokaryotic organism, fungi, or protozoa. The term also includes microbes that are pathogenic for humans, animals, or plants. The term further includes either Gram-negative and Gram-positive bacteria as well as bacteria that are neither Gram-negative nor Gram-positive. Accordingly, any bacteria, fungi, protozoa or other organism that is shown to become resistant to antibiotics is appropriate for use in the claimed methods.

As used herein, the term “ascorbic compound” includes any pharmaceutically acceptable salt of ascorbate, including sodium ascorbate, as well as calcium ascorbate, magnesium ascorbate, ascorbyl palmitate and ascorbic acid itself and a combination thereof. The term “lysine” refers to lysine in its electrically neutral form or a pharmaceutically acceptable salt of lysine which includes lysine hydrochloride, lysine dihydrochloride, lysine succinate, lysine glutamate, lysine orotate as well as L-Lysine. The term “proline” refers to proline and proline in a pharmaceutical acceptable salt of proline which includes proline hydrochloride, proline glutamate, as well as L-proline. The term “arginine” refers to arginine and arginine in a pharmaceutical acceptable salt of arginine which includes arginine hydrochloride, arginine glutamate, as well as L-arginine. The term “polyphenols” includes, but is not limited to, Epigallocatechin Gallate (EGCg), epicatechin gallate (ECG), epigallocatechin, epicatechin, and catechin which is an anti-oxidant polyphenol isolated from green tea, Red Wine Polyphenols (RWP) derived from grapes, and polyphenols derived generally from fruits and vegetables.

As used herein, the term “pharmaceutically acceptable salt” refers to those salts which retain the biological effectiveness and properties of the active ingredient of the biochemical composition, which are not otherwise undesirable. Pharmaceutically acceptable salts include, but are not limited to, salts of sodium, potassium, calcium, magnesium, aluminum and the like.

As used herein, the term “an effective amount” refers to that an amount of the biochemical composition disclosed in this application that when administered to an individual subject in need thereof, is sufficient to reduce the antimicrobial resistance of microbes thereby enhancing the antimicrobial activity of drugs. An effective amount may be determined routinely by one of ordinary skill in the art in view of this disclosure and the knowledge in the art. For example, one could start doses at levels lower than that required to achieve a desired therapeutic effect and gradually increase the dosage until the desired effect is achieved.

The term “microbe that is resistant to one or more drugs” as used herein includes microbes that are characterized by mutations in a gene that is the target of a drug. It also includes microbes that are characterized by mutations in multiple genes that affect drug resistance. The term “mutation” includes an alteration of at least one nucleotide in the sequence of a nucleic acid molecule in a microbe that is capable of influencing drug resistance. Such a mutation can result in altered gene regulation in the microbe or in the expression of an altered polypeptide.

The term, “pharmaceutically acceptable means” as used herein means a pharmaceutically-acceptable material, composition or vehicle, such as a liquid or solid filler, diluent, excipient, solvent or encapsulating material, involved in carrying or transporting the antimicrobial agents or compounds of the invention from one organ, or portion of the body, to another organ, or portion of the body without affecting its biological effect. Each carrier should be “acceptable” in the sense of being compatible with the other ingredients of the composition and not injurious to the subject.

The preferred route of administration is oral, using a convenient daily dosage regimen which can be adjusted according to the degree of severity of the disease state to be treated. For such oral administration, a pharmaceutically acceptable composition containing the compounds of the invention, or a pharmaceutically acceptable salt thereof, is formed by the incorporation of any of the normally employed. Such compositions take the form of solutions, suspensions, tablets, pills, capsules, powders, sustained release formulations and the like.

Additional embodiments and advantages of the invention will be set forth in the description that follows, and in part will be obvious from the description, or may be learned by practice of the invention. This invention may be realized and obtained by means of the composition and method of treatment particularly pointed out from the description and drawings, and from the claims.

A composition of can contain at least one ascorbic compound selected from the group consisting of ascorbic acid, pharmaceutically acceptable ascorbate salts, calcium ascorbate, magnesium ascorbate, ascorbyl palmitate and/or mixtures thereof in combination with lysine hydrochloride, L-lysine or pharmaceutically acceptable lysine salts, L-proline, proline hydrochloride or pharmaceutically acceptable proline salts, L-arginine, arginine hydrochloride, or pharmaceutically acceptable argine salts, polyphenols thereof and/or mixtures of these compounds. The composition can be effective in reducing the antimicrobial resistance of microbes.

Preferably, a composition of biochemical substances comprises about 400 mg to about 1500 mg of L-lysine. More preferably, a composition of biochemical substances comprises about 700 mg to about 1200 mg of L-lysine. Most preferably, a composition of biochemical substances comprises about 1000 mg of L-lysine.

Preferably, a composition of biochemical substances comprises about 500 mg to about 1500 mg of L-proline. More preferably, a composition of biochemical substances comprises about 600 mg to about 1000 mg of L-proline. Most preferably, a composition of biochemical substances comprises about 750 mg of L-proline.

Preferably, a composition of biochemical substances comprises about 200 mg to about 1000 mg of L-arginine. More preferably, a composition of biochemical substances comprises about 300 mg to about 800 mg of L-arginine. Most preferably, a composition of biochemical substances comprises about 500 mg of L-arginine.

Preferably, a composition of biochemical substances comprises about 400 mg to about 1500 mg of ascorbic acid. More preferably, a composition of biochemical substances comprises about 600 mg to about 1000 mg of ascorbic acid. Most preferably, a composition of biochemical substances comprises about 710 mg of ascorbic acid.

Preferably, a composition of biochemical substances comprises about 5 mg to about 50 mg of calcium. More preferably, a composition of biochemical substances comprises about 10 mg to about 40 mg of calcium. Most preferably, a composition of biochemical substances comprises about 22 mg of calcium.

Preferably, a composition of biochemical substances comprises about 10 mg to about 100 mg of magnesium. More preferably, a composition of biochemical substances comprises about 30 mg to about 70 mg of magnesium. Most preferably, a composition of biochemical substances comprises about 50 mg of magnesium.

Preferably, a composition of biochemical substances comprises about 500 mg to about 2000 mg of polyphenols. More preferably, a composition of biochemical substances comprises about 600 mg to about 1200 mg of polyphenols. Most preferably, a composition of biochemical substances comprises about 800 mg of polyphenols.

Preferably, a composition of biochemical substances comprises about 100 mg to about 500 mg of N-acetyl-cysteine. More preferably, a composition of biochemical substances comprises about 150 mg to about 300 mg of N-acetyl-cysteine. Most preferably, a composition of biochemical substances comprises about 200 mg of N-acetyl-cysteine.

Preferably, a composition of biochemical substances comprises about 5 μg to about 60 μg of selenium. More preferably, a composition of biochemical substances comprises about 10 μg to about 40 μg of selenium. Most preferably, a composition of biochemical substances comprises about 30 μg of selenium.

Preferably, a composition of biochemical substances comprises about 0.5 mg to about 5 mg of copper. More preferably, a composition of biochemical substances comprises about 1 mg to about 3 mg of copper. Most preferably, a composition of biochemical substances comprises about 2 mg of copper.

Preferably, a composition of biochemical substances comprises about 0.5 mg to about 3 mg of manganese. More preferably, a composition of biochemical substances comprises about 0.75 mg to about 2 mg of manganese. Most preferably, a composition of biochemical substances comprises about 1 mg of manganese.

Preferably, a composition of biochemical substances comprises L-lysine, L-proline, L-arginine, ascorbic acid and polyphenols. The composition of biochemical substances may be administered with a drug.

More preferably, a composition of biochemical substances comprises L-lysine, L-proline, L-arginine, ascorbic acid, calcium, magnesium, polyphenols, N-acetyl-cysteine, selenium, copper and manganese and is administered with a drug.

Most preferably, a composition of biochemical substances consists essentially of L-lysine, L-proline, L-arginine, ascorbic acid, calcium, magnesium, polyphenols, N-acetyl-cysteine, selenium, copper and manganese and is administered with a drug.

Preferably, a composition of biochemical substances is administered with an antibiotic. Most preferably, a composition of biochemical substances is administered with a drug selected from the group consisting essentially of Ciprofloxacin (CIP), a Trimethoprim-Sulfamethoaxzole (SXT), a Cefotaxime (CTX), a Gentamicin (GM), an Ofloxacin (OFX), a Nalidixic acid (NA), an Ampicillin (AMP), a Vancomycin (V), a Tetracycline (TE), a Rifampicin (RA), a Doxycycline (D), a Ceftriaxone (Cro), and a Kanamycin (K).

The composition may include a conventional pharmaceutically acceptable means or excipient and a compound of the invention as the active agent, and in addition, may include other medicinal agents, pharmaceutical agents, carriers, adjuvant, etc.

These compositions may also contain additional agents, such as preservatives, wetting agents, emulsifying agents and dispersing agents. Prevention of the action of microorganisms may be ensured by the inclusion of various antibacterial and antifungal agents. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Pharmaceutical compositions of the present invention may be administered to epithelial surfaces of the body orally, parenterally, topically, rectally, nasally, intravaginally, intracisternally. They are of course given by forms suitable for each administration route. For example, they are administered in tablets or capsule form, by injection, inhalation, eye lotion, ointment, etc., administration by injection, infusion or inhalation; topical by lotion or ointment; and rectal or vaginal suppositories.

The phrases “parenteral administration” and “administered parenterally” as used herein means modes of administration other than enteral and topical administration, usually by injection.

In some methods, the compositions of the invention can be administered peripherally. The phrases “administered peripherally” as used herein means the administration of an antibacterial or a contraceptive agent, drug or other material other than directly into the central nervous system, such that it enters the subject's system and, thus, is subject to metabolism and other like processes.

In some methods, the compositions of the invention can be topically administered to any epithelial surface. An “epithelial surface” according to this invention is defined as an area of tissue that covers external surfaces of a body, or that lines hollow structures including, but not limited to, cutaneous and mucosal surfaces.

Compositions can be formulated in a variety of conventional forms employed for topical administration. These include, for example, semi-solid and liquid dosage forms, such as liquid solutions or suspensions, suppositories, douches, enemas, gels, creams, emulsions, lotions, slurries, powders, sprays, lipsticks, foams, pastes, toothpastes, ointments, salves, balms, drops, troches, chewing gums, lozenges, mouthwashes and rinses. Conventionally used carriers for topical applications include for example pectin, gelatin and derivatives thereof, polylactic acid and polyglycolic acid polymers or copolymers thereof.

Such compositions can be particularly useful, for example, for treatment or prevention of an unwanted cell, vaginal Neisseria gonorrhea, or infections of the oral cavity, including cold sores, infections of eye, the skin, or the lower intestinal tract. Standard composition strategies for topical agents can be applied to the antimicrobial compounds, or pharmaceutically acceptable salts thereof in order to enhance the persistence and residence time of the drug, and to improve the prophylactic efficacy achieved.

For topical application in the lower intestinal tract or vaginally, a rectal suppository, a suitable enema, a gel, an ointment, a solution, a suspension or an insert can be used. Topical transdermal patches may also be used. Transdermal patches have the added advantage of providing controlled delivery of the compositions of the invention to the body. Such dosage forms can be made by dissolving or dispersing the agent in a proper medium.

Compositions of the invention can be administered in the form of suppositories for rectal or vaginal administration. These can be prepared by mixing the agent with a suitable non-irritating carrier which is solid at room temperature but liquid at rectal temperature and therefore will melt in the rectum or vagina to release the drug.

For ophthalmic applications, the pharmaceutical compositions can be formulated as micronized suspensions in isotonic, pH adjusted sterile saline, or, preferably, as solutions in isotonic, pH-adjusted sterile saline, either with or without a preservative such as benzylalkonium chloride.

Powders and sprays can contain carriers such as lactose, talc, silicio acid, aluminum hydroxide, calcium silicates and polyamide powder, or mixtures of these substances. Sprays can additionally contain customary propellants.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants. Aerosols generally are prepared from isotonic solutions.

Sterile injectable forms of the compositions of this invention can be aqueous or oleaginous suspensions. These suspensions may be formulated according to techniques known in the art using suitable dispersing or wetting agents and suspending agents. Wetting agents, emulsifiers and lubricants, as well as coloring agents, release agents, coating agents, sweetening, flavoring and perfuming agents, preservatives and antioxidants can also be present in the compositions.

For preventive applications, the pharmaceutical composition of the invention can be applied prior to physical contact with a microbe. The timing of application prior to physical contact can be optimized to maximize the prophylactic effectiveness of the compound. The timing of application will vary depending on the mode of administration, the epithelial surface to which it is applied, the surface area, doses, the stability and effectiveness of composition under the pH of the epithelial surface, the frequency of application, e.g., single application or multiple applications. One skilled in the art will be able to determine the most appropriate time interval required to maximize prophylactic effectiveness of the compound.

As used in this application, the singular form “a,” “an,” and “the” include plural references unless the context clearly dictates otherwise. For example, the term “an agent” includes a plurality of agents, including mixtures thereof.

The term, “mammal” as used herein means any of various warm-blooded vertebrate animals of the class Mammalia, including humans, characterized by a covering of hair on the skin and, in the female, milk-producing mammary glands for nourishing the young.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., Short Protocols in Molecular Biology, 3rd Ed., ed. by Ausubel, F. et al. Wiley, N.Y. (1995), Methods In Enzymology Academic Press, Inc., N.Y., Immunochemical Methods In Cell And Molecular Biology Mayer and Walker, eds., Academic Press, London (1987), Handbook Of Experimental Immunology, Volumes I-IV D. M. Weir and C. C. Blackwell, eds. (1986), all of which are herein incorporated in their entirety by reference for all purposes.

The following specific examples are provided as a guide to assist in the practice of the invention, and are not intended as a limitation on the scope of the invention.

### EXAMPLES

### Example 1

This example illustrates the preparation of a representative composition containing the biochemical compounds listed in the following table (Table 1).

TABLE 1

| Biochemical Substances | Units | Amount |
|---|---|---|
| L-Lysine | mg | 1000 |
| L-Proline | mg | 750 |
| L-Arginine | mg | 500 |
| Ascorbic Acid | mg | 710 |
| Calcium | mg | 22 |
| Magnesium | mg | 50 |
| Polyphenols | mg | 1000 |
| N-acetyl-cysteine | mg | 200 |
| Selenium | μg | 30 |
| Copper | mg | 2 |
| Manganese | mg | 1 |

mg = milligram
μg = microgram

### Example 2

To demonstrate the utility of the composition of the invention as the therapeutic and preventive agents for enhancing the antimicrobial activity of a drug, we evaluate the composition of these biochemical compounds for its ability to increase the diameter of the inhibition zone of the bacterial colonies and for its ability to increase the susceptibility of the bacterial colonies.

The bacterial colonies (Salmonella) were exposed to the composition in accordance with the invention comprising list of components and amounts during the exponential growth state of the Salmonella bacteria in the presence of antibiotics. Thereafter, the effect of the composition to the bacterial colonies was quantified by comparing the post-treatment state of colonies to their pre-treatment state. Collectively, the results indicate that the composition was effective in increasing the inhibition zone of the bacterial colonies and in increasing the bacterial colonies' susceptibility to the antibiotics as compared to control.

### The results of the test are listed in Table 2

TABLE 2
Data of Salmonella colonies after exposing to composition listed in Table 1
being in the presence of various antibiotics.

| Salmonella | CIP | | SXT | | CTX | | GM | | OFX | |
|---|---|---|---|---|---|---|---|---|---|---|
| (30 colonies) | 1 h | 2 h | 1 h | 2 h | 1 h | 2 h | 1 h | 2 h | 1 h | 2 h |
| Colonies | 29 | 27 | 24 | 23 | 22 | 26 | 27 | 26 | 29 | 29 |
| showing | | | | | | | | | | |
| increasing | | | | | | | | | | |
| inhibition | | | | | | | | | | |
| zones | | | | | | | | | | |
| Colonies | 1 | 2 | 4 | 4 | 5 | 4 | 0 | 2 | 0 | 0 |
| with no | | | | | | | | | | |
| change in | | | | | | | | | | |
| inhibition | | | | | | | | | | |
| zone. | | | | | | | | | | |
| Colonies | 0 | 1 | 1 | 2 | 1 | 0 | 3 | 2 | 1 | 1 |
| showing | | | | | | | | | | |
| decreasing | | | | | | | | | | |
| inhibition | | | | | | | | | | |
| zone. | | | | | | | | | | |
| % colonies | 96.7 | 90.0 | 80.0 | 76.7 | 80.0 | 86.7 | 90.0 | 86.7 | 96.7 | 96.7 |
| showing | | | | | | | | | | |
| increase in | | | | | | | | | | |
| inhibition | | | | | | | | | | |
| zone | | | | | | | | | | |
| Colonies | 16 | 15 | 12 | 12 | 16 | 16 | 17 | 17 | 15 | 14 |
| showing | | | | | | | | | | |
| change in | | | | | | | | | | |
| susceptibility | | | | | | | | | | |
| % of | 53.3 | 50.0 | 40.0 | 40.0 | 53.3 | 53.3 | 56.7 | 56.7 | 50.0 | 46.7 |
| colonies | | | | | | | | | | |
| showing | | | | | | | | | | |
| change in | | | | | | | | | | |
| susceptibility | | | | | | | | | | |

The percentages of colonies showing increase in their susceptibility was derived by dividing the number of colonies that became more susceptible with the total number of originally resistant colonies.

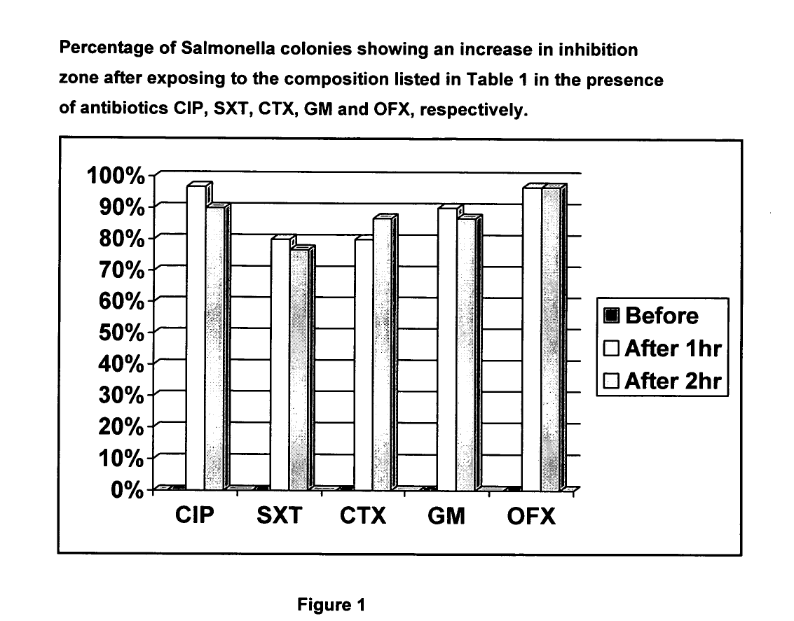

The percentages of colonies that show increase in their inhibition zone were calculated by dividing the number of colonies that showed an increase in the diameter of their inhibition zone (including those that were originally sensitive and still sensitive after the increase) with the total of 30 tested colonies Results of the study, as illustrated in FIG. 1 demonstrate that the treatment of cultures of bacterial colonies with the composition affected their antimicrobial resistance pattern and resulted in increasing the diameter of the inhibition zone. Accordingly, the results proves that antimicrobial affect of the antibiotics was enhanced by the composition.

FIG. 1 is a graph of result from a laboratory experiment conducted using Salmonella colonies after exposing the colonies to the biochemical composition listed in Table 1 in a laboratory experiment in the presence of antibiotics, CIP, SXT, CTX, GM and OFX.

The change to the inhibition zone of each colony was recorded at two time points of 1 and 2 hours during the exponential growth phase. Averages of 88.7% of colonies were shown to have increased their inhibition zone.

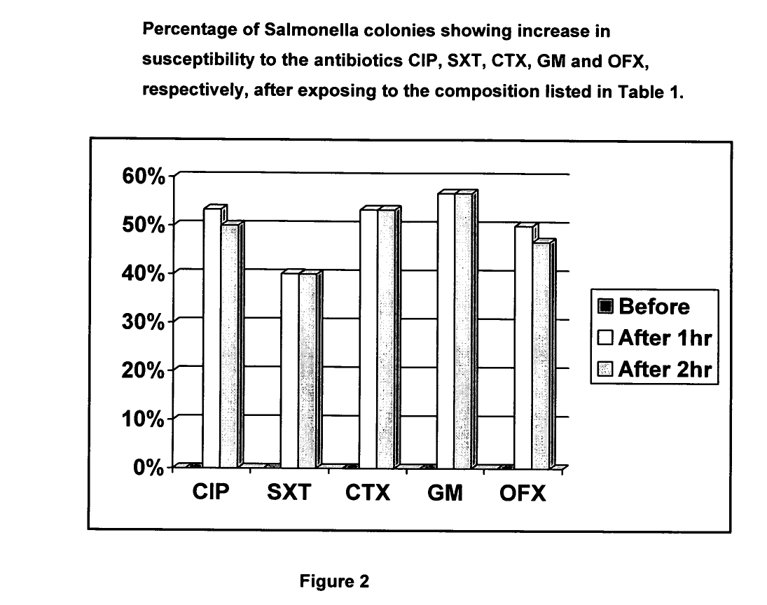

FIG. 2 is a graph of the percentage of Salmonella colonies showing increase in susceptibility after the bacterial colonies were exposed to one of the antibiotics, CIP, SXT, CTX, GM, and OFX during the exponential stage of growth and subsequently treated with the composition listed in Table 1. The percentage of colonies reflecting an increase in susceptibility was determined by dividing the number of colonies that became susceptible by the total number of resistant colonies. In FIG. 2, the results show that an average of 45% of the colonies became increasingly susceptible to the antimicrobial activity of the antimicrobial agent in combination with a biochemical composition in accordance with the invention.

### Example 3

The bacterial colonies (S. saprophyticus) were tested according to same procedures outlined in Example 2.

### The results of the test are listed in Table 3

TABLE 3
Data of S. saprophyticus colonies after exposing to composition
listed in Table 1 being in the presence of various antibiotics.

| | GM | | V | | TE | |
|---|---|---|---|---|---|---|
| S. saprophyticus (15 colonies) | 1 h | 2 h | 1 h | 2 h | 1 h | 2 h |
| Colonies showing an increase | 14 | 15 | 8 | 10 | 8 | 8 |
| in inhibition zone | | | | | | |
| Colonies with no change in | 1 | 0 | 5 | 4 | 7 | 7 |
| inhibition zone. | | | | | | |
| Colonies showing a decrease | 0 | 0 | 2 | 1 | 0 | 0 |
| in inhibition zone. | | | | | | |
| Percent colonies showing | 93.3 | 100.0 | 53.3 | 66.7 | 53.3 | 53.3 |
| increase in inhibition zone | | | | | | |
| (%). | | | | | | |

The percentages of colonies showing increase in their susceptibility was derived by dividing the number of colonies that became more susceptible with the total number of originally resistant colonies.

The percentages of colonies that show increase in their inhibition zone were calculated by dividing the number of colonies that showed an increase in the diameter of their inhibition zone (including those that were originally sensitive and still sensitive after the increase) with the total of 15 tested colonies

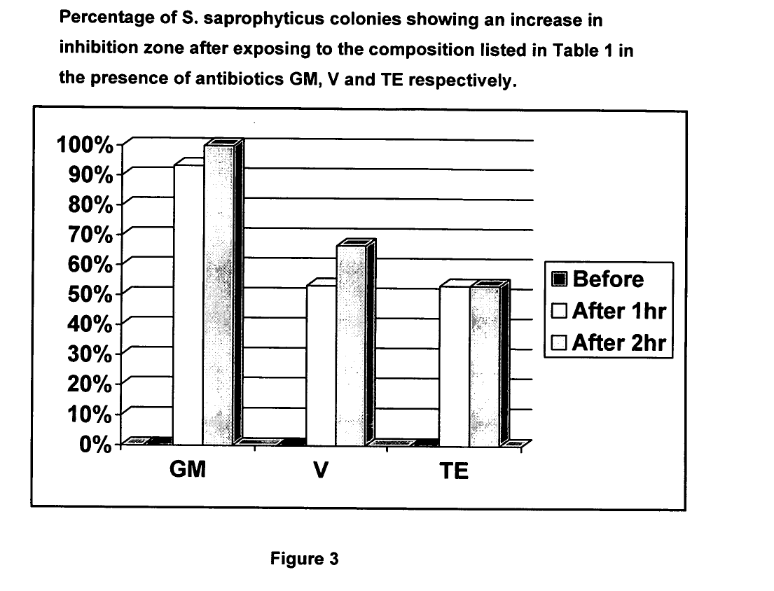

Results of the study, as illustrated in FIG. 3 demonstrate that the treatment of cultures of bacterial colonies with the composition affected their antimicrobial resistance pattern and resulted in increasing the diameter of the inhibition zone.

FIG. 3 is a graph of result from a laboratory experiment conducted using S. saprophyticus colonies exposed to one of the antibiotics, GM, V and TE and treated with the biochemical composition containing the compounds listed in Table 1 in a laboratory experiment.

The change to the inhibition zone of each colony was recorded at two time points of 1 and 2 hours during the exponential growth phase. Averages of 70% of colonies were shown to have increased their inhibition zone.

### Example 4

The bacterial colonies (S. aureus) were tested according to same procedures outlined in Example 2.

### The results of the test are listed in Table 4

TABLE 4
Data of S. aureus colonies after exposing to composition listed
in Table 1 being in the presence of antibiotic GM.

GM

| S. aureus (15 colonies) | 1 h | 2 h |
|---|---|---|
| Colonies showing an increase in | 14 | 14 |
| inhibition zone | | |
| Number of colonies with no change | 1 | 0 |
| in inhibition zone. | | |
| Colonies showing a decrease in | 0 | 1 |
| inhibition zone. | | |
| Percent colonies showing increase | 93.3 | 93.3 |
| in inhibition zone (%). | | |

The percentages of colonies showing increase in their susceptibility was derived by dividing the number of colonies that became more susceptible with the total number of originally resistant colonies.

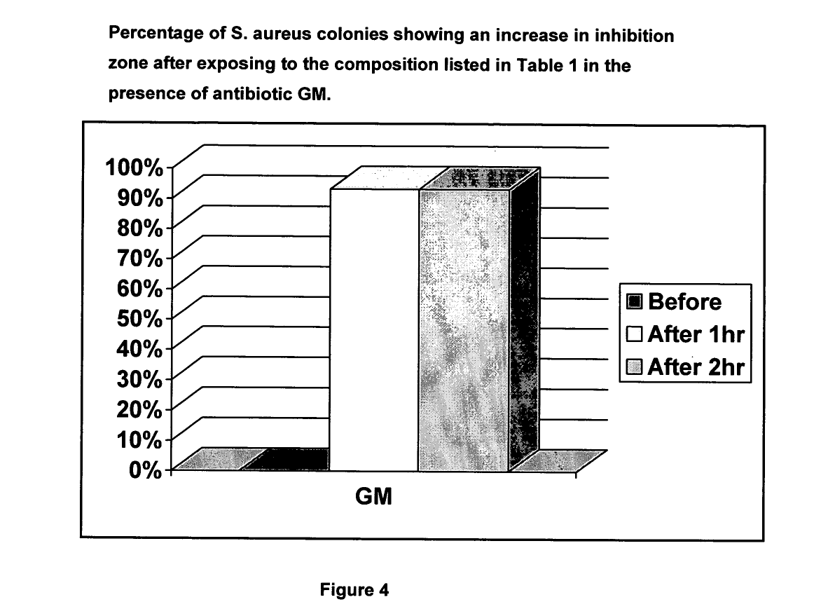

The percentages of colonies that show increase in their inhibition zone were calculated by dividing the number of colonies that showed an increase in the diameter of their inhibition zone (including those that were originally sensitive and still sensitive after the increase) with the total of 15 tested colonies FIG. 4 is a graph of result from a laboratory experiment conducted using S. aureus colonies exposed to the antibiotic GM and treated with the biochemical composition containing the compounds listed in Table 1 in a laboratory experiment.

The change to the inhibition zone of each colony was recorded at two time points of 1 and 2 hours during the exponential growth phase. Averages of 93.3% of colonies were shown to have increased their inhibition zone.

Results of the study, as illustrated in FIG. 4 demonstrate that the treatment of cultures of bacterial colonies with the composition affected their antimicrobial resistance pattern and resulted in increasing the diameter of the inhibition zone.

### Example 5

The bacterial colonies (Listeria) were tested according to same procedures outlined in Example 2.

### The results of the test are listed in Table 5.

TABLE 5
Data of Listeria colonies after exposing to composition listed
in Table 1 being in the presence of various antibiotics.

| Listeria (30 | TE | | AMP | | GM | | SXT | |
|---|---|---|---|---|---|---|---|---|
| colonies) | 1 h | 2 h | 1 h | 2 h | 1 h | 2 h | 1 h | 2 h |
| Colonies | 26 | 26 | 20 | 21 | 26 | 28 | 30 | 30 |
| showing | | | | | | | | |
| increasing | | | | | | | | |
| inhibition zone | | | | | | | | |
| Number of | 2 | 2 | 4 | 3 | 1 | 0 | 0 | 0 |
| colonies with | | | | | | | | |
| no change in | | | | | | | | |
| inhibition zone. | | | | | | | | |
| Colonies | 2 | 2 | 6 | 6 | 3 | 2 | 0 | 0 |
| showing | | | | | | | | |
| a decreasing | | | | | | | | |
| inhibition zone. | | | | | | | | |
| Percent | 86.7 | 86.7 | 66.7 | 70.0 | 86.7 | 93.3 | 100 | 100 |
| colonies | | | | | | | | |
| showing | | | | | | | | |
| increase in | | | | | | | | |
| inhibition zone | | | | | | | | |
| (%). | | | | | | | | |

The percentages of colonies showing increase in their susceptibility was derived by dividing the number of colonies that became more susceptible with the total number of originally resistant colonies.

The percentages of colonies that show increase in their inhibition zone were calculated by dividing the number of colonies that showed an increase in the diameter of their inhibition zone (including those that were originally sensitive and still sensitive after the increase) with the total of 30 tested colonies

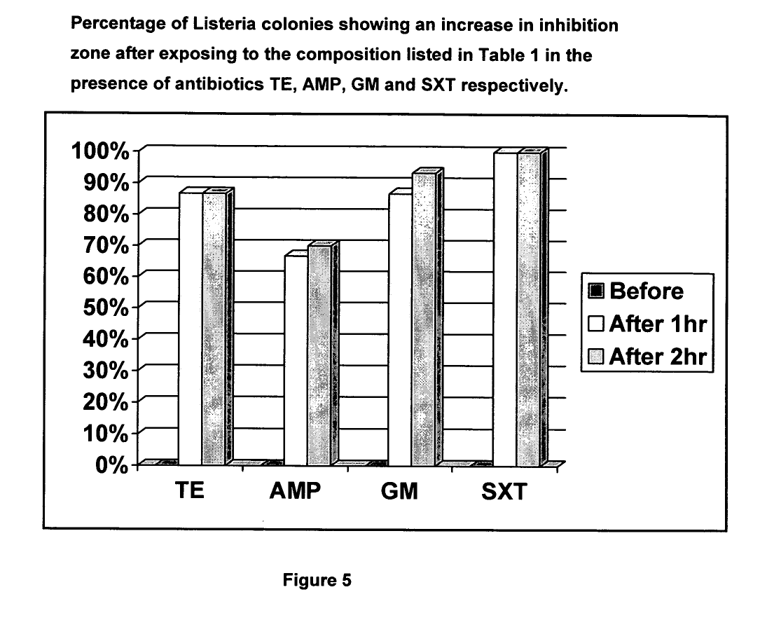

FIG. 5 is a graph of result from a laboratory experiment conducted using Listeria colonies exposed to one of the antibiotics, TE, AMP, GM, SXT and treated with the biochemical composition containing the compounds listed in Table 1 in a laboratory experiment.

The change to the inhibition zone of each colony was recorded at two time points of 1 and 2 hours during the exponential growth phase. Averages of 86.3% of colonies were shown to have increased their inhibition zone.

Accordingly, results of the study, as illustrated in FIG. 5 demonstrate that the treatment of cultures of bacterial colonies with the composition affected their antimicrobial resistance pattern and resulted in increasing the diameter of the inhibition zone.

### Example 6

The bacterial colonies (Brucella) were tested according to same procedures outlined in Example 2.

### The results of the test are listed in Table 6

TABLE 6
Data of Brucella colonies after exposing to the composition listed in Table 1
being in the presence of various antibiotics.

| Brucella | | | | | | | | | | |
|---|---|---|---|---|---|---|---|---|---|---|
| (30 | GM | | TE | | SXT | | RA | | Cro | |
| colonies) | 1 h | 2 h | 1 h | 2 h | 1ZZ1 h | 2 h | 1 h | 2 h | 1 h | 2 h |
| Colonies | 22 | 14 | 20 | 20 | 30 | 30 | 30 | 27 | 27 | 27 |
| showing | | | | | | | | | | |
| increasing | | | | | | | | | | |
| inhibition | | | | | | | | | | |
| zone | | | | | | | | | | |
| Colonies | 4 | 3 | 10 | 9 | 0 | 0 | 0 | 0 | 3 | 3 |
| with no | | | | | | | | | | |
| change in | | | | | | | | | | |
| inhibition | | | | | | | | | | |
| zone. | | | | | | | | | | |
| Colonies | 4 | 13 | 0 | 1 | 0 | 0 | 0 | 3 | 0 | 0 |
| showing | | | | | | | | | | |
| decreasing | | | | | | | | | | |
| inhibition | | | | | | | | | | |
| zone. | | | | | | | | | | |
| Percent | 73.3 | 46.7 | 66.7 | 66.7 | 100 | 100 | 100 | 90.0 | 90.0 | 90.0 |
| colonies | | | | | | | | | | |
| showing | | | | | | | | | | |
| increase | | | | | | | | | | |
| in | | | | | | | | | | |
| inhibition | | | | | | | | | | |
| zone (%). | | | | | | | | | | |

The percentages of colonies showing increase in their susceptibility was derived by dividing the number of colonies that became more susceptible with the total number of originally resistant colonies.

The percentages of colonies that show increase in their inhibition zone were calculated by dividing the number of colonies that showed an increase in the diameter of their inhibition zone (including those that were originally sensitive and still sensitive after the increase) with the total of 30 tested colonies

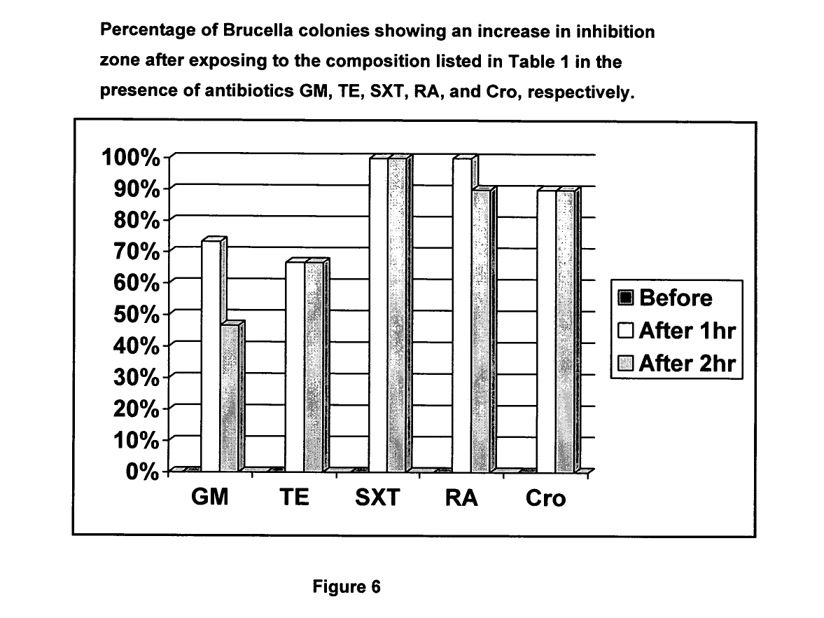

FIG. 6 is a graph of result from a laboratory experiment conducted using Brucella colonies exposed to one of the antibiotics, GM, TE, SXT, RA, Cro and treated with the biochemical composition containing the compounds listed in Table 1 in a laboratory experiment.

The change to the inhibition zone of each colony was recorded at two time points of 1 and 2 hours during the exponential growth phase. Averages of 82.3% of colonies were shown to have increased their inhibition zone.

In yet another test result as demonstrated in FIG. 6, the treatment of cultures of bacterial colonies with the composition affected their antimicrobial resistance pattern and resulted in increasing the diameter of the inhibition zone.

### Example 7

The bacterial colonies (Yerssinia) were tested according to same procedures outlined in Example 2.

### The results of the test are listed in Table 7

TABLE 7
Data of Yerssinia colonies after exposing to the composition listed in Table 1
being in the presence of various antibiotics.

| Yerssinia | | | | | | | | | | | | |
|---|---|---|---|---|---|---|---|---|---|---|---|---|
| (30 | GM | | OFX | | SXT | | NA | | K | | CIP | |
| colonies) | 1 h | 2 h | 1 h | 2 h | 1 h | 2 h | 1 h | 2 h | 1 h | 2 h | 1 h | 2 h |
| Colonies | 24 | 27 | 27 | 24 | 17 | 16 | 18 | 22 | 23 | 24 | 29 | 22 |
| showing | | | | | | | | | | | | |
| increase | | | | | | | | | | | | |
| in | | | | | | | | | | | | |
| inhibition | | | | | | | | | | | | |
| zone | | | | | | | | | | | | |
| Colonies | 3 | 2 | 0 | 1 | 12 | 8 | 7 | 4 | 5 | 2 | 1 | 8 |
| with no | | | | | | | | | | | | |
| change in | | | | | | | | | | | | |
| inhibition | | | | | | | | | | | | |
| zone. | | | | | | | | | | | | |
| Colonies | 3 | 1 | 3 | 5 | 1 | 6 | 5 | 4 | 2 | 4 | 0 | 6 |
| showing | | | | | | | | | | | | |
| decrease | | | | | | | | | | | | |
| in | | | | | | | | | | | | |
| inhibition | | | | | | | | | | | | |
| zone. | | | | | | | | | | | | |
| Percent | 80.0 | 90.0 | 90.0 | 80.0 | 56.7 | 53.3 | 60.0 | 73.3 | 76.7 | 80.0 | 96.7 | 73.3 |
| colonies | | | | | | | | | | | | |
| showing | | | | | | | | | | | | |
| increase | | | | | | | | | | | | |
| in | | | | | | | | | | | | |
| inhibition | | | | | | | | | | | | |
| zone (%). | | | | | | | | | | | | |

The percentages of colonies showing increase in their susceptibility was derived by dividing the number of colonies that became more susceptible with the total number of originally resistant colonies.

The percentages of colonies that show increase in their inhibition zone were calculated by dividing the number of colonies that showed an increase in the diameter of their inhibition zone (including those that were originally sensitive and still sensitive after the increase) with the total of 30 tested colonies

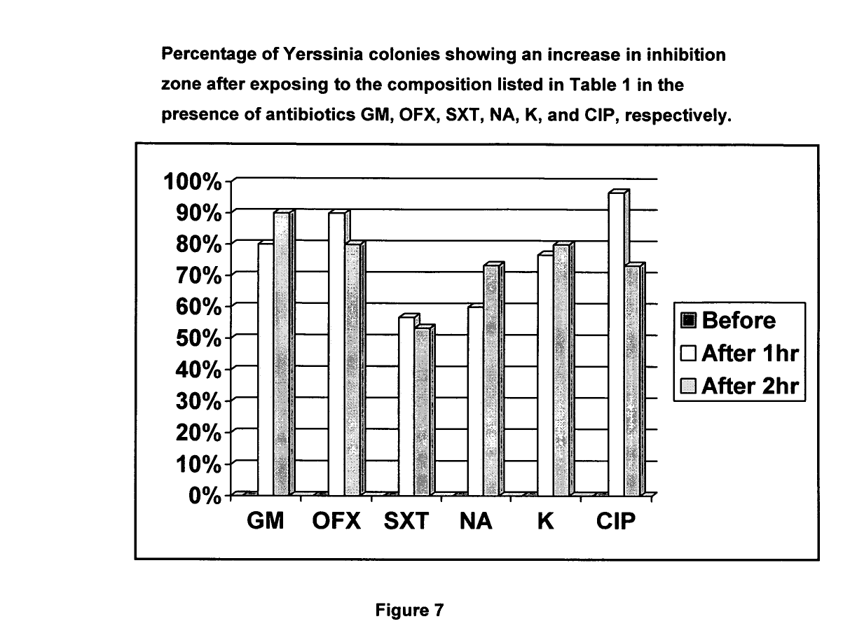

FIG. 7 is a graph of result from a laboratory experiment conducted using Yerssinia colonies exposed to one of the antibiotic, GM, OFX, SXT, NA, K, CIP and treated with the biochemical composition containing the compounds listed in Table 1 in a laboratory experiment.

The change to the inhibition zone of each colony was recorded at two time points of 1 and 2 hours during the exponential growth phase. Averages of 75.8% of colonies were shown to have increased their inhibition zone.

As demonstrated in FIG. 7, the treatment of cultures of bacterial colonies with the composition affected their antimicrobial resistance pattern and resulted in increasing the diameter of the inhibition zone.

### Example 8

The bacterial colonies (E. coli) were tested according to same procedures outlined in Example 2.

### The results of the test are listed in Table 8

TABLE 8
Data of E coli colonies after exposing to composition listed in Table 1
being in the presence of various antibiotics.

| | CTX | | GM | | OFX | |
|---|---|---|---|---|---|---|
| E. coli (30 colonies) | 1 h | 2 h | 1 h | 2 h | 1 h | 2 h |
| Number of colonies | 26 | 22 | 22 | 12 | 20 | 17 |
| showing an increase | | | | | | |
| in inhibition zone | | | | | | |
| Number of colonies | 1 | 3 | 2 | 14 | 5 | 5 |
| with no change in | | | | | | |
| inhibition zone. | | | | | | |
| Number of colonies | 3 | 5 | 6 | 6 | 5 | 8 |
| showing a decrease in | | | | | | |
| inhibition zone. | | | | | | |
| Percent colonies | 86.7 | 73.3 | 73.3 | 40.0 | 66.7 | 56.7 |
| showing increase in | | | | | | |
| inhibition zone (%). | | | | | | |

The percentages of colonies showing increase in their susceptibility was derived by dividing the number of colonies that became more susceptible with the total number of originally resistant colonies.

The percentages of colonies that show increase in their inhibition zone were calculated by dividing the number of colonies that showed an increase in the diameter of their inhibition zone (including those that were originally sensitive and still sensitive after the increase) with the total of 30 tested colonies

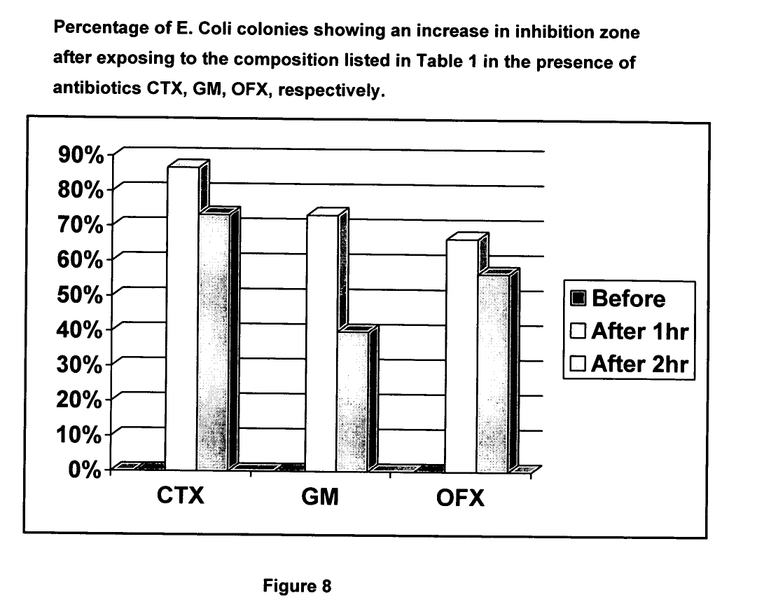

FIG. 8 is a graph of result from a laboratory experiment conducted using E. coli colonies exposed to one of the antibiotic, CTX, GM, OFX and treated with the biochemical composition containing the compounds listed in Table 1 in a laboratory experiment.

The change to the inhibition zone of each colony was recorded at two time points of 1 and 2 hours during the exponential growth phase. Averages of 66.1% of colonies were shown to have increased their inhibition zone.

As demonstrated in FIG. 8, the treatment of cultures of bacterial colonies with the composition affected their antimicrobial resistance pattern and resulted in increasing the diameter of the inhibition zone.

As such, we are able to deduce from the experimental results that bacteria colonies grown in the presence of the composition in Table 1 lose their resistance to antimicrobial activity. When the chemical compounds of the present invention are combined, reduction of antimicrobial resistance occurs. This combined effect is surprising in its effectiveness in enhancing the antimicrobial activity of a drug, in its ability to reduce the antibiotic resistance of bacteria and in its ability to treat infection. The increase in the bacterial colonies' inhibition zone and the increase in their susceptibility demonstrate a full therapeutic effect of the biochemical composition. Accordingly, based on the experimental results, one of skill in the art would recognize that application of a biochemical compound in accordance with the invention, in conjunction with administration of one or more antimicrobial agents will be effective in the treating and preventing health conditions caused by an infection.

While the present invention has been described with reference to specific embodiments thereof, it should be understood by those skilled in the art that various changes may be made and equivalents may be substituted without departing from the true spirit and scope of the invention. One of ordinary skills in the art would appreciate that the effective amounts of the biochemical compounds may vary depending on the variations in patients, durations of treatment, etc. Modifications may be made to adapt a particular situation, and composition of matter. A number of embodiments of the invention have been described in the present application; nevertheless, it will be understood all such modifications are intended to be within the scope of the following claims.

The contents of all references, pending patent applications and published patents, cited throughout this application are hereby expressly incorporated by reference.

### CONCLUSION

It is to be understood that the above description is intended to be illustrative and not restrictive. Those skilled in the art will recognize, or be able to ascertain using no more than routine experimentation, many equivalents to the specific embodiments of the invention described herein. Such equivalents are intended to be encompassed by the following claims.

What is claimed is:

1. A method for treating an infection comprising the step of administering to a mammal an effective amount of a composition comprising lysine, proline, arginine, ascorbic compound, and polyphenols.

2. The method according to claim 1, wherein the composition further comprises calcium, magnesium, N-acetyl-cysteine, selenium, copper, and manganese.

3. The method according to claim 2, wherein the composition comprising about 400 to about 1500 mg lysine, about 500 to about 1500 mg proline, about 200 to about 1000 mg arginine, about 400 to about 1500 mg of ascorbic compound, about 5 to about 50 mg calcium, about 10 to about 100 mg magnesium, about 500 to about 2000 mg polyphenols, about 100 to about 500 mg N-acetyl-cysteine, about 5 to about 60 μg selenium, about 0.5 to about 5 mg copper, and about 0.5 to about 3 mg manganese.

4. The method according to claim 2, wherein the composition comprising about 1000 mg lysine, about 750 m proline, about 500 mg arginine, about 710 mg of ascorbic compound, about 22 mg calcium, about 50 mg magnesium, about 800 mg polyphenols, about 200 mg N-acetyl-cysteine, about 30 μg selenium, about 2 mg copper, and about 1 mg manganese.

5. A method for preventing or treating a health condition caused by an infection comprising the step of administering to a mammal a drug and an effective amount of a composition comprising lysine, proline, arginine, ascorbic compound, and polyphenols.

6. The method according to claim 5, wherein the composition further comprises calcium, magnesium, N-acetyl-cysteine, selenium, copper, and manganese.

7. The method according to claim 6, wherein the composition comprising about 400 to about 1500 mg lysine, about 500 to about 1500 mg proline, about 200 to about 1000 mg arginine, about 400 to about 1500 mg of ascorbic compound, about 5 to about 50 mg calcium, about 10 to about 100 mg magnesium, about 500 to about 2000 mg polyphenols, about 100 to about 500 mg N-acetyl-cysteine, about 5 to about 60 μg selenium, about 0.5 to about 5 mg copper, and about 0.5 to about 3 mg manganese.

8. The method according to claim 6, wherein the composition comprising about 1000 mg lysine, about 750 m proline, about 500 mg arginine, about 710 mg of ascorbic compound, about 22 mg calcium, about 50 mg magnesium, about 800 mg polyphenols, about 200 mg N-acetyl-cysteine, about 30 μg selenium, about 2 mg copper, and about 1 mg manganese.

9. The method according to claim 5, where the drug is an antibiotic.

10. The method according to claim 9, wherein the antibiotic is selected from a group consisting of Ciprofloxacin, Trimethoprim-Sulfamethoaxzole, Cefotaxime, Gentamicin, Ofloxacin, Nalidixic acid, Ampicillin, Vancomycin, Tetracycline, Rifampicin, Doxycycline, Ceftriaxone, and a Kanamycin.

11. The method according to claim 5, wherein the composition is in the form of tablets, pills, injections, infusions, inhalations, suppositories or other pharmaceutically acceptable means of delivery.

12. A method for enhancing antimicrobial activity of a drug comprising treating a microbe that is resistant to one or more drugs with a drug to which the microbe is resistant to and a composition in the effective amount comprising lysine, proline, arginine, ascorbic compound, and polyphenols.

13. The method according to claim 12, wherein the composition further comprises lysine, calcium, magnesium, N-acetyl-cysteine, selenium, copper, and manganese.

14. The method according to claim 13, wherein the composition comprising about 400 to about 1500 mg lysine, about 500 to about 1500 mg proline, about 200 to about 1000 mg arginine, about 400 to about 1500 mg of ascorbic compound, about 5 to about 50 mg calcium, about 10 to about 100 mg magnesium, about 500 to about 2000 mg polyphenols, about 100 to about 500 mg N-acetyl-cysteine, about 5 to about 60 μg selenium, about 0.5 to about 5 mg copper, and about 0.5 to about 3 mg manganese.

15. The method according to claim 13, wherein the composition comprising about 1000 mg lysine, about 750 m proline, about 500 mg arginine, about 710 mg of ascorbic compound, about 22 mg calcium, about 50 mg magnesium, about 800 mg polyphenols, about 200 mg N-acetyl-cysteine, about 30 μg selenium, about 2 mg copper, and about 1 mg manganese.

16. The method according to claim 12, where the drug is an antibiotic.

17. The method according to claim 16, wherein the antibiotic is selected from a group consisting of Ciprofloxacin, Trimethoprim-Sulfamethoaxzole, Cefotaxime, Gentamicin, Ofloxacin, Nalidixic acid, Ampicillin, Vancomycin, Tetracycline, Rifampicin, Doxycycline, Ceftriaxone, and a Kanamycin.

18. The method according to claim 12, wherein the composition is in the form of tablets, pills, injections, infusions, inhalations, suppositories or other pharmaceutically acceptable means of delivery.

19. A pharmaceutical composition comprising an antibiotic and a composition in the effective amount comprising lysine, proline, arginine, ascorbic compound, calcium, magnesium, polyphenols, N-acetyl-cysteine, selenium, copper, and manganese.

20. The composition according claim 19, wherein the antibiotic is selected from a group consisting of Ciprofloxacin, Trimethoprim-Sulfamethoaxzole, Cefotaxime, Gentamicin, Ofloxacin, Nalidixic acid, Ampicillin, Vancomycin, Tetracycline, Rifampicin, Doxycycline, Ceftriaxone, Teicoplanin and a Kanamycin.

21. The composition according to claim 19, wherein the composition is in the form of tablets, pills, injections, infusions, inhalations, suppositories or other pharmaceutically acceptable means of delivery
